(19) United States
(12) Patent Application Publication
(10) Pub. No.: US 2015/0045254 A1
(43) Pub. Date: Feb. 12, 2015

(54) SYSTEMS, METHODS AND DEVICES FOR ELECTROCHEMICAL DETECTION USING HELPER OLIGONUCLEOTIDES

(71) Applicant: XAGENIC INC., Toronto (CA)

(72) Inventor: Graham D. Jack, Toronto (CA)

(21) Appl. No.: 14/454,652

(22) Filed: Aug. 7, 2014

Related U.S. Application Data
(60) Provisional application No. 61/863,280, filed on Aug. 7, 2013.

(51) Int. Cl. C12Q 1/68

(52) U.S. Cl. CPC C12Q 1/6825
USPC 506/9; 435/6.11; 435/5; 435/287.2; 506/39

(57) ABSTRACT

Disclosed herein are systems, devices, and methods for the electrochemical detection of a target using a helper oligonucleotide (each a helper oligo, or collectively, helper oligos).

### CROSS-REFERENCE TO RELATED APPLICATION

This application claims priority to U.S. Provisional Application No. 61/863,280 filed Aug. 7, 2013, which is hereby incorporated by reference herein in its entirety.

### SEQUENCE LISTING

The instant application contains a Sequence Listing which has been submitted electronically in ASCII format and is hereby incorporated by reference in its entirety. Said ASCII copy, created on Aug. 7, 2014, is named 109904-0013-101_SL.txt and is 1,329 bytes in size.

### BACKGROUND

The development of low cost, high throughput sensors that can detect nucleic acid targets with high sensitivity and specificity is highly desirable. Many standard diagnostic assays, such as cell cultures and genetic testing with PCR amplification, require sending samples to labs and have long turnaround times of several days or weeks. Many patients, in such cases, do not return to the care provider to receive the results or treatments, and in some cases, the long turn-around can compromise the ability to properly treat the condition. While rapid, point-of-care assays are in development, in such systems it is difficult to achieve the high sensitivity and specificity necessary to reduce the occurrence of false positive and false negative results. Thus, alternative systems and methods for increasing sensitivity and specificity could be beneficial for improved point-of-care applications.

### SUMMARY

Disclosed herein are systems, devices, and methods for the electrochemical detection of a target using a helper oligonucleotide (each a helper oligo, or collectively, helper oligos). According to one aspect, helper oligos (negatively charged amino acid sequences) are used to increase the amount of charge present upon hybridization to a target molecule by a capture molecule in order to increase sensitivity of electrochemical detection. In some implementations, a helper oligo may hybridize to a portion of an RNA/DNA. The RNA/DNA target may then bind to an electrode-bound PNA probe of reverse complimentarily. Once bound to the probe, the entire complex (target and helper sequences) increases the overall charge at the surface of the electrode, which increases the magnitude of a detected signal. When bound to the target, helper oligos can increase the surface charge and detection sensitivity more so than if a target binds to the probe in the absence of the helper oligo. Helper oligos can also be tagged or linked to charged moieties, which further increases charge upon hybridization with the target. In addition, helper oligos, when applied a sample containing the target prior to detection, can open up areas of the DNA/RNA target that may be partially inaccessible to the probe, thus allowing for more efficient binding of the probe to a desired sequence within the target. Moreover, by opening up the target sequences, the helper oligo may reduce the effects of non-specific binding by increasing accessibility to the target sequence.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 depicts a schematic of the electrochemical detection of a target according to some implementations;

FIG. 2 depicts an electrochemical readout indicating the presence/absence of a target according to some implementations;

FIG. 3 depicts a nanostructured microelectrode-based electrochemical detector according to some implementations;

FIGS. 4A, 4B, 4C, and 4D depict a schematic of electrochemical detection using a helper oligo according to some implementations;

FIGS. 5A and 5B depict the enzymatic extension of a helper oligo according to some implementations;

FIGS. 6A, 6B, and 6C depict the enzymatic extension of a helper oligo using rolling circle amplification according to some implementations;

FIGS. 7A and 7B depict a helper oligo tagged with a charged moiety according to some implementations;

FIGS. 8A and 8B depict a helper oligo tagged with a branched oligonucleotide structure according to some implementations;

FIGS. 9A, 9B, 9C, and 9D depict test results in accordance with an illustrative embodiment;

FIG. 10 depicts a chamber for performing electrochemical detection using a helper oligo according to some implementations;

FIG. 11 depicts an illustrative processing for detecting a target using a helper oligo;

FIG. 12 depicts a cartridge system for receiving, preparing, and analyzing a biological sample according to some implementations;

FIG. 13 depicts a cartridge for an analytical detection system according to some implementations; and

FIG. 14 depicts an automated testing system according to some implementations.

### DETAILED DESCRIPTION

To provide an overall understanding of the systems, devices, and methods described herein, certain illustrative implementations will be described. It is to be understood that the systems, devices, and methods disclosed herein, while shown for use in diagnostic systems for the detection of biological disease markers, may be applied to other systems that require multiplexed electrochemical analysis.

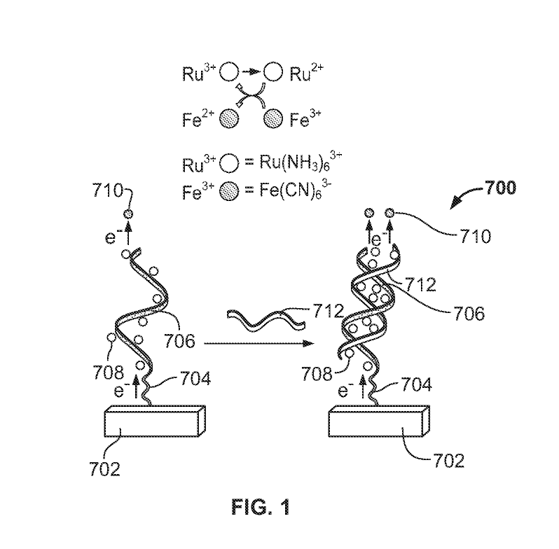

FIGS. 1-4 depict illustrative tools, sensors, biosensors, and techniques for detecting target analytes, including cellular, molecular, or tissue components, by electrochemical methods. FIG. 1 depicts electrochemical detection of a nucleotide strand using a biosensor system. System 700 includes an electrode 702 with an associated probe 706 attached to the electrode 702 via a linker 704. The probe 706 is a molecule or group of molecules, such as nucleic acids (e.g., DNA, RNA, cDNA, mRNA, rRNA, etc.), oligonucleotides, peptide nucleic acids (PNA), locked nucleic acids, proteins (e.g., antibodies, enzymes, etc.), or peptides, that is able to bind to or otherwise interact with a biomarker target (e.g., receptor, ligand) to provide an indication of the presence of the ligand or receptor in a sample. The linker 704 is a molecule or group of molecules which tethers the probe 706 to the electrode 702, for example, through a chemical bond, such as a thiol bond.

In some implementations, the probe 706 is a polynucleotide capable of binding to a target nucleic acid sequence through one or more types of chemical bonds, such as complementary base pairing and hydrogen bond formation. This binding is also called hybridization or annealing. For example, the probe 706 may include naturally occurring nucleotide and nucleoside bases, such as adenine (A), guanine (G), cytosine (C), thymine (T), and uracil (U), or modified bases, such as 7-deazaguanosine and inosine. The bases in probe 706 can be joined by a phosphodiester bond (e.g., DNA and RNA molecules), or with other types of bonds. For example, the probe 706 can be a peptide nucleic acid (PNA) oligomer in which the constituent bases are joined by peptide bonds rather than phosphodiester linkages. A peptide nucleic acid (PNA) oligomer may contain a backbone comprised of N-(2-aminoethyl)-glycine units linked by peptide bonds. Peptide nucleic acids have a higher binding affinity and increased specificity to complementary nucleic acid oligomers, and accordingly, may be particularly beneficial in diagnostic and other sensing applications, as described herein.

In some implementations, the probe 706 has a sequence partially or completely complementary to a target marker 712, such as a nucleic acid sequence sought. Target marker 712 is a molecule for detection, as will be described in further detail below. In some implementations, probe 706 is a single-stranded oligonucleotide capable of binding to at least a portion of a target nucleic acid sought to be detected. In certain approaches, the probe 706 has regions which are not complementary to a target sequence, for example, to adjust hybridization between strands or to serve as a non-sense or negative control during an assay. The probe 706 may also contain other features, such as longitudinal spacers, double-stranded regions, single-stranded regions, poly(T) linkers, and double stranded duplexes as rigid linkers and PEG spacers. In certain approaches, electrode 702 can be configured with multiple, different probes 706 for multiple, different targets 712.

The probe 706 includes a linker 704 that facilitates binding of the probe 706 to the electrode 702. In certain approaches, the linker 704 is associated with the probe 706 and binds to the electrode 702. For example, the linker 704 may be a functional group, such as a thiol, dithiol, amine, carboxylic acid, or amino group. For example, it may be 4-mercaptobenzoic acid coupled to a 5′ end of a polynucleotide probe. In certain approaches, the linker 704 is associated with the electrode 702 and binds to the probe 706. For example, the electrode 702 may include an amine, silane, or siloxane functional group. In certain approaches, the linker 704 is independent of the electrode 702 and the probe 706. For example, linker 704 may be a molecule in solution that binds to both the electrode 702 and the probe 706.

Under appropriate conditions, such as in a suitable hybridization buffer, the probe 706 can hybridize to a complementary target marker 712 to provide an indication of the presence of target marker 712 in a sample. In certain approaches, the sample is a biological sample from a biological host. For example, a sample may be tissue, cells, proteins, fluid, genetic material, bacterial matter or viral matter, plant matter, animal matter, cultured cells, or other organisms or hosts. The sample may be a whole organism or a subset of its tissues, cells or component parts, and may include cellular or noncellular biological material. Fluids and tissues may include, but are not limited to, blood, plasma, serum, cerebrospinal fluid, lymph, tears, saliva, blood, mucus, lymphatic fluid, synovial fluid, cerebrospinal fluid, amniotic fluid, amniotic cord blood, urine, vaginal fluid, semen, tears, milk, and tissue sections. The sample may contain nucleic acids, such as deoxyribonucleic acids (DNA), ribonucleic acids (RNA), or copolymers of deoxyribonucleic acids and ribonucleic acids or combinations thereof. In certain approaches, the target marker 712 is a nucleic acid sequence that is known to be unique to the host, pathogen, disease, or trait, and the probe 706 provides a complementary sequence to the sequence of the target marker 712 to allow for detection of the host sequence in the sample.

In certain aspects, systems, devices and methods are provided to perform processing steps, such as purification and extraction, on the sample. Analytes or target molecules for detection, such as nucleic acids, may be sequestered inside of cells, bacteria, or viruses. The sample may be processed to separate, isolate, or otherwise make accessible, various components, tissues, cells, fractions, and molecules included in the sample. Processing steps may include, but are not limited to, purification, homogenization, lysing, and extraction steps. The processing steps may separate, isolate, or otherwise make accessible a target marker, such as the target marker 712 in or from the sample.

In certain approaches, the target marker 712 is genetic material in the form of DNA or RNA obtained from any naturally occurring prokaryotes such, pathogenic or non-pathogenic bacteria (e.g., Escherichia, Salmonella, Clostridium, Chlamydia, etc.), eukaryotes (e.g., protozoans, parasites, fungi, and yeast), viruses (e.g., Herpes viruses, HIV, influenza virus, Epstein-Barr virus, hepatitis B virus, etc.), plants, insects, and animals, including humans and cells in tissue culture. Target nucleic acids from these sources may, for example, be found in biological samples of a bodily fluid from an animal, including a human. In certain approaches, the sample is obtained from a biological host, such as a human patient, and includes non-human material or organisms, such as bacteria, viruses, other pathogens.

A target nucleic acid molecule, such as target marker 712, may optionally be amplified prior to detection. The target nucleic acid can be in a double-stranded or single-stranded form. A double-stranded form may be treated with a denaturation agent to render the two strands into a single-stranded form, or partially single-stranded form, at the start of the amplification reaction, by methods such as heating, alkali treatment, or by enzymatic treatment.

Once the sample has been treated to expose a target nucleic acid, e.g., target molecule 712, the sample solution can be tested as described herein to detect hybridization between probe 706 and target molecule 712. For example, electrochemical detection may be applied as will be described in greater detail below. If target molecule 712 is not present in the sample, the systems, device, and methods described herein may detect the absence of the target molecule. For example, in the case of diagnosing a bacterial pathogen, such as Chlamydia trachomatis (CT), the presence in the sample of a target molecule, such as an RNA sequence from Chlamydia trachomatis, would indicate presence of the bacteria in the biological host (e.g., a human patient), and the absence of the target molecule in the sample indicates that the host is not infected with Chlamydia trachomatis. Similarly, other markers may be used for other pathogens and diseases.

Referring to FIG. 1, the probe 706 of the system 700 hybridizes to a complementary target molecule 712. In certain approaches, the hybridization is through complementary base pairing. In certain approaches, mismatches or imperfect hybridization may also take place. “Mismatch” typically refers to pairing of noncomplementary nucleotide bases between two different nucleic acid strands (e.g., probe and target) during hybridization. Complementary pairing is commonly accepted to be A-T, A-U, and C-G. Conditions of the local environment, such as ionic strength, temperature, and pH can effect the extent to which mismatches between bases may occur, which may also be termed the “specificity” or the “stringency” of the hybridization. Other factors, such as the length of a nucleotide sequence and type of probe, can also affect the specificity of hybridization. For example, longer nucleic acid probes have a higher tolerance for mismatches than shorter nucleic acid probes.

As illustrated in the figures, the presence or absence of target marker 712 in the sample is determined through electrochemical techniques. These electrochemical techniques allow for the detection of extremely low levels of nucleic acid molecules, such as a target RNA molecule obtained from a biological host. Applications of electrochemical techniques are described in further detail in U.S. Pat. Nos. 7,361,470 and 7,741,033, and PCT Application No. PCT/US12/024015, which are hereby incorporated by reference herein in their entireties. A brief description of these techniques, as applied to the current system, is provided below, it being understood that the electrochemical techniques are illustrative and non-limiting and that other techniques can be envisaged for use with the other systems, devices and methods of the current system.

In the electrochemical application of FIG. 1, a solution sample is applied to the working electrode 702. In practice, a redox pair having a first transition metal complex 708 and a second transition metal complex 710 is added to the sample solution. A signal generator or potentiostat is used to apply an electrical signal to the working electrode 702, causing the first transition metal complex 708 to change oxidative states, due to its close association with the working electrode 702 and the probe 706. Electrons can then be transferred to the second transition metal complex 710, creating a current through the working electrode 702, through the sample, and back to the signal generator. The current signal is amplified by the presence of the first transition metal complex 708 and the second transition metal complex 710, as will be described below.

The first transition metal complex 708 and the second transition metal complex 710 together form an electrochemical reporter system which amplifies the signal. A transition metal complex is a structure composed of a central transition metal atom or ion, generally a cation, surrounded by a number of negatively charged or neutral ligands possessing lone pairs of electrons that can be transferred to the central transition metal. A transition metal complex (e.g., complexes 708 and 710) includes a transition metal element found between the Group IIA elements and the Group IIB elements in the periodic table. In certain approaches, the transition metal is an element from the fourth, fifth, or sixth periods between the Group IIA elements and the Group IIB elements of the periodic table of elements. In some implementations, the first transition metal complex 708 and second transition metal complex 710 include a transition metal selected from the group comprising cobalt, iron, molybdenum, osmium, ruthenium and rhenium. In some implementations, the ligands of the first transition metal complex 708 and second transition metal complex 710 is selected from the group comprising pyridine-based ligands, phenathroline-based ligands, heterocyclic ligands, aquo ligands, aromatic ligands, chloride (Cl), ammonia (NH₃⁺), or cyanide (CN⁻). In certain approaches, the first transition metal complex 108 is a transition metal ammonium complex. For example, as shown in FIG. 1, the first transition metal complex 108 is Ru(NH₃)₆³⁺. In certain approaches, the second transition metal complex 710 is a transition metal cyanate complex. For example, as shown in FIG. 1, the second transition metal complex is Fe(CN)₆³⁻. In certain approaches, the second transition metal complex 710 is an iridium chloride complex such as IrCl₆²⁻ or IrCl₆³⁻.

In certain applications, if the target molecule 712 is present in the sample solution, the target molecule 712 will hybridize with the probe 706, as shown on the right side of FIG. 1. The first transition metal complex 108 (e.g., Ru(NH₃)1+) is cationic and accumulates, due to electrostatic attraction forces as the nucleic acid target molecule 712 hybridizes at the probe 706. The second transition metal complex 710 (e.g., Fe(CN)1−) is anionic and is repelled from the hybridized target molecule 712 and probe 706. A signal generator, such as a potentiostat, is used to apply a voltage signal to the electrode. As the signal is applied, the first transition metal complex 708 is reduced (e.g., from Ru(NH₃)₁₊ to Ru(NH₃)₁₋). The reduction of the second metal complex 710 (e.g., Fe(CN)₆³) is more thermodynamically favorable, and accordingly, electrons (e⁻) are shuttled from the reduced form of the first transition metal complex 708 to the second transition metal complex 710 to reduce the second transition metal complex (e.g., Fe(CN)₆³⁻ to Fe(CN)₆⁴⁻) and regenerate the original first transition metal complex 708 (e.g., Ru(NH₃)₆³⁺). This catalytic shuttling process allows increased electron flow through the working electrode 702 when the potential is applied, and amplifies the response signal (e.g., a current), when the target molecule 712 is present. When the target molecule 712 is absent from the sample, the measured signal is significantly reduced.

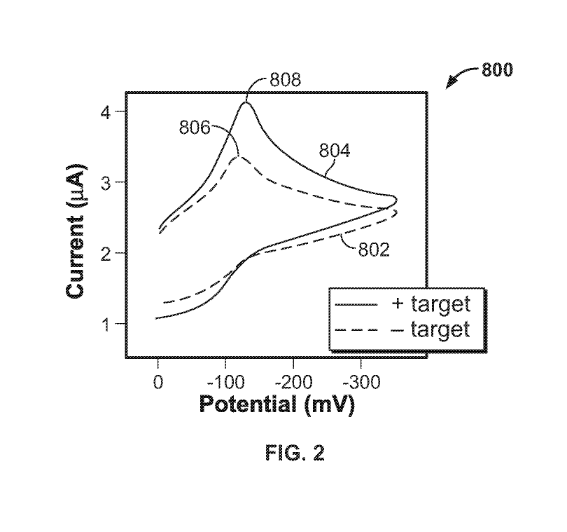

Chart 800 of FIG. 2 depicts representative electrochemical detection signals. A signal generator such as a potentiostat, is used to apply a voltage signal at an electrode, such as working electrode 702 of FIG. 1. Electrochemical techniques including, but not limited to cyclic voltammetry, amperometry, chronoamperometry, differential pulse voltammetry, calorimetry, and potentiometry may be used for detecting a target marker. In certain approaches, an applied potential or voltage is altered over time. For example, the potential may be cycled or ramped between two voltage points, such from 0 mV to −300 mV and back to 0 mV, while measuring the resultant current. Accordingly, chart 800 depicts the current along the vertical axis at corresponding potentials between 0 mV and −300 mV, along the horizontal axis. Data graph 802 represent a signal measured at an electrode, such as working electrode 702 of FIG. 1, in the absence of a target marker. Data graph 804 represents a signal measured at an electrode, such as working electrode 702 of FIG. 1, in the presence of a target marker. As can be seen on data graph 804, the signal recorded in the presence of the target molecule provides a higher amplitude current signal, particularly when comparing peak 808 with peak 806 located at approximately −100 mV. Accordingly, the presence and absence of the marker can be differentiated.

In certain applications, a single electrode or sensor is configured with two or more probes, arranged next to each other, or on top of or in close proximity within the chamber so as to provide target and control marker detection in an even smaller point-of-care size configuration. For example, a single electrode sensor may be coupled to two types of probes, which are configured to hybridize with two different markers. In certain approaches, a single probe is configured to hybridize and detect two markers. In certain approaches, two types of probes may be coupled to an electrode in different ratios. For example, a first probe may be present on the electrode sensor at a ratio of 2:1 to the second probe. Accordingly, the sensor is capable of providing discrete detection of multiple analytes. For example, if the first marker is present, a first discrete signal (e.g., current) magnitude would be generated, if the second marker is present, a second discrete signal magnitude would be generated, if both the first and second marker are present, a third discrete signal magnitude would be generated, and if neither marker is present, a fourth discrete signal magnitude would be generated. Similarly, additional probes could also be implemented for increased numbers of multi-target detection.

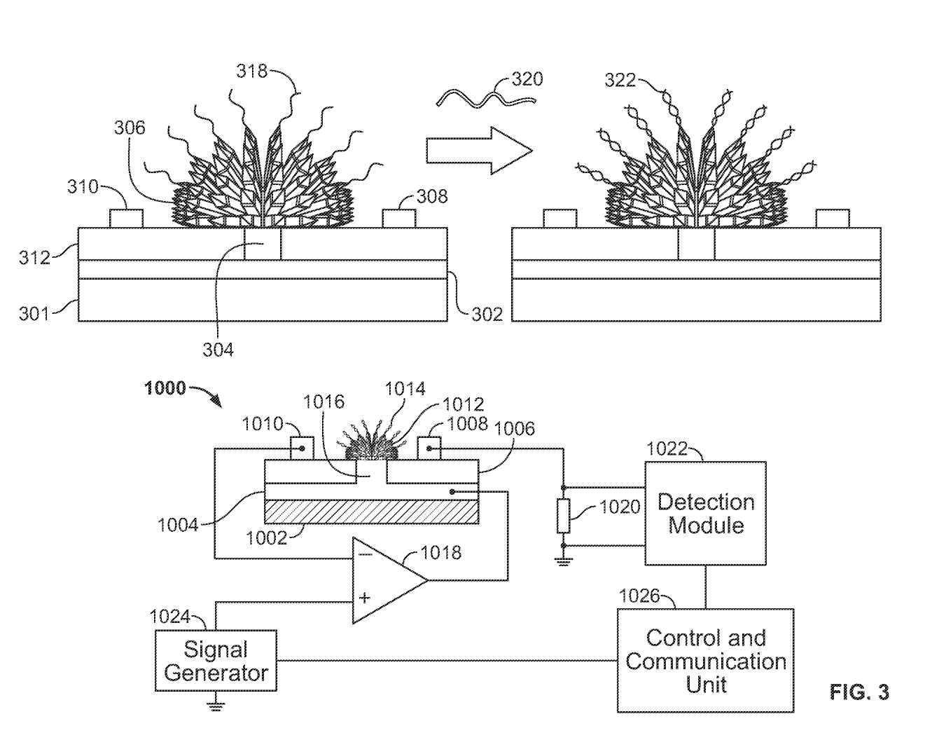

FIG. 3 depicts a detection system using a nanostructured microelectrode for electrochemical detection of a nucleotide strand, in accordance with an implementation. Nanostructured microelectrodes are microscale electrodes with nanoscale features. Nanostructured microelectrode systems are described in further detail in U.S. application Ser. No. 13/061,465, U.S. Pat. Nos. 7,361,470 and 7,741,033, and PCT Application No. PCT/US12/024015, which are hereby incorporated by reference herein in their entireties. Functionalized detection unit 1000 utilizes a nanostructured microelectrode as a working electrode, which increases the sensitivity of the system by dramatically increasing the surface-area of the working electrode. Probe 318 is tethered to working electrode 306 along with other probes that are chemically identical to probe 318, using any suitable method described herein. Probe 318 is specific to target marker 320, and may be any suitable type of probe, such as a PNA probe. Probe 318 may be tethered to working electrode 306 using any suitable method. For example, nitrogen containing nanostructured microelectrodes (e.g., TiN, WN, or TaN) can bind with an amine functional group of probe 318. Upon introduction of target marker 320 into the sample well, complex 322 may be formed by selective binding of target marker 320 with probe 318. Electrochemical reagents may be pre-mixed with the sample upon application to the sample well. In some implementations, the sample is flushed from the sample wells after a time interval has passed to allow binding of target marker 320 with probe 318, and a solution containing electrochemical reagents is then added to the sample well to enable electrochemical detection.

FIG. 3 also shows an exemplary system for detecting a target marker in accordance with the various implementations described herein. The detection system 1000 includes solid support 1002, lead 1004, aperture layer 1006, counter electrode 1008, reference electrode 1010, and working electrode 1012, which extends from lead 1002 through aperture 1016. However, any suitable configuration of electrodes may be used. If the sample contains a target marker of interest, complex 1014 may form on the surface of working electrode 1012.

The detection system 1000 shown in FIG. 3 incorporates an illustrative three-electrode potentiostat configuration, however it should be understood that any suitable configuration of components may be used, and the terminals of the potentiostat may be coupled to the various electrodes in any suitable manner. Lead 1004 is connected to the output terminal of control amplifier 1018. Counter electrode 1008 is connected to resistor 1020, which is grounded. It should be understood, however, that resistor 1020 does not necessarily need to be grounded. Detection module 1022 is connected across resistor 1020, which is operable to determine a current through resistor 1020 based on a measured potential and the value of resistance. The detection module 1022 may be configured to provide real-time current measurement in response to any input waveform. Reference electrode 100 is connected to the inverting terminal of control amplifier 1018. Signal generator 1024 is connected to the non-inverting terminal of control amplifier 1018. This configuration maintains constant potential at the working electrode while allowing for accurate measurements of the current.

Control and communication unit 1026 is operably coupled to detection module 1022 and signal generator 1024. Control and communication unit 1026 may synchronize the input waveforms and output measurements, and may receive and store the input and output in a memory. In some implementations, control and communication unit 1026 is a separate unit that interfaces with a detection system. For example, detection system 1000 may be a disposable cartridge with a plurality of input and output terminals that can interface with control and communication unit 1026. In some implementations, control and communication unit 1026 is operably coupled to a display unit that displays the output as a function of input. In some implementations, control and communication unit 1026 transmits the input and output information to a remote destination for storage and display. For example, control and communication unit 1026 could be a mobile device or capable of being interfaced with a mobile device. In some implementations, control and communication unit 1026 provides power to the detection system 1000. Detection system 1000 may be powered using any suitable power source, including a battery or a plugged-in AC power source.

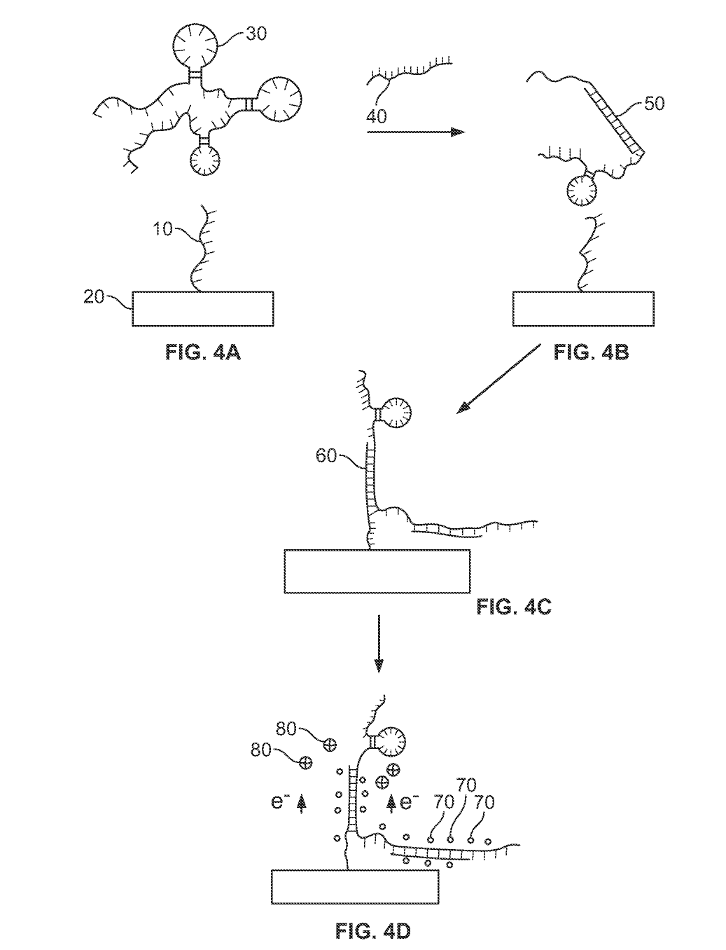

FIGS. 4A-C show an illustrative embodiment a detection strategy using a helper oligo. In FIG. 4A, a probe 10 may be affixed to an electrode 20, such as a planar electrode or a nanostructured microelectrode. The probe may be, for example, a single-stranded PNA or single-stranded DNA probe. Any suitable linker may be used to affix probe 10 to electrode 20. The probe is specific to a complementary sequence portion of a target 30. Target 30 may be, for example, a RNA or DNA from CT. The target may self-hybridize and form hairpin loops or other secondary structures that may cause steric hindrance, thus making the complementary sequence portion difficult to access by the probe. In some embodiments, a helper oligo 40 is contacted with a sample containing or suspected of containing target 30 prior to contacting the sample with the probe. In some embodiments, the sample may be simultaneously contacted with probe 10 and helper oligo 40. FIG. 4B shows the formation of a first complex 50 formed as a result of helper oligo 40 hybridizing to target 30. The hybridization of helper oligo 40 to target 30 may eliminate secondary structures and make a portion of target 30 more rigid and accessible to the surrounding solvent, thereby “opening up” target 30. In FIG. 4C, complex 50 is brought into contact with probe 10, forming complex 60. Complex 60 is formed as a result of the hybridization of probe 10 with the complementary sequence portion of target 30. In some embodiments, the region to which the helper oligo hybridizes with the target may be selected such that a terminal end of the helper oligo, that is closest to a base-pair formed between the probe and target, is closer to a surface-bound terminal end of the probe than to a non-surface-bound terminal end of the probe, thereby localizing the hybridized helper oligo near electrode 20. FIG. 4D shows the introduction of an electrochemical application for detecting the target, similar to the scheme illustrated in FIG. 1. A first transition metal complex 70 and a second transition metal complex 80, for example, may be utilized to amplify the signal. The signal may be detected using any suitable method described herein. The detected signal will be greatly amplified due to the additional charge provided by the helper oligo near the surface of electrode 20.

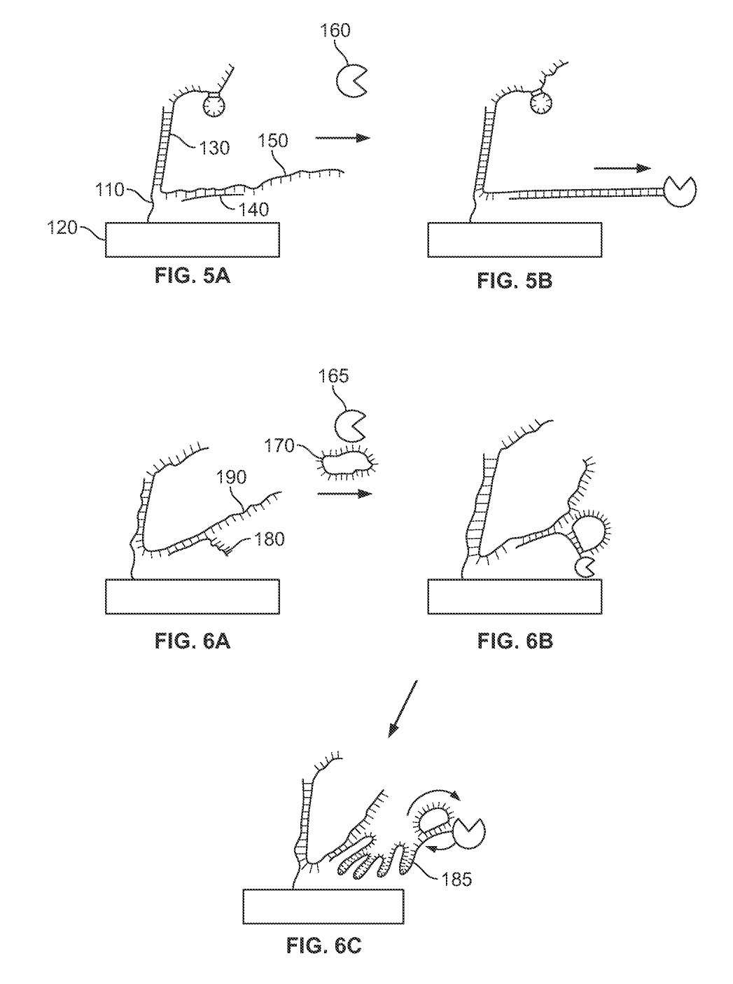

FIGS. 5A and B show the enzymatic extension of a helper oligo 140 to further enhance the detected signal. FIG. 5A shows a complex formed from a target 130 simultaneously hybridized to a probe 110 and helper oligo 140, and bound to electrode 120 by a linker attached to probe 110. Target 130 may have a tail region 150 that is not hybridized to the helper oligo. FIG. 5B shows the enzymatic extension of helper oligo 140 when enzyme 160 is applied to the complex. In some embodiments, enzyme 160 may be a DNA or RNA polymerase. Enzyme 160 binds to a terminal end of helper oligo 140 and polymerizes helper oligo 140 along tail region 150 until helper oligo 140 is extended to the length of tail region 150.

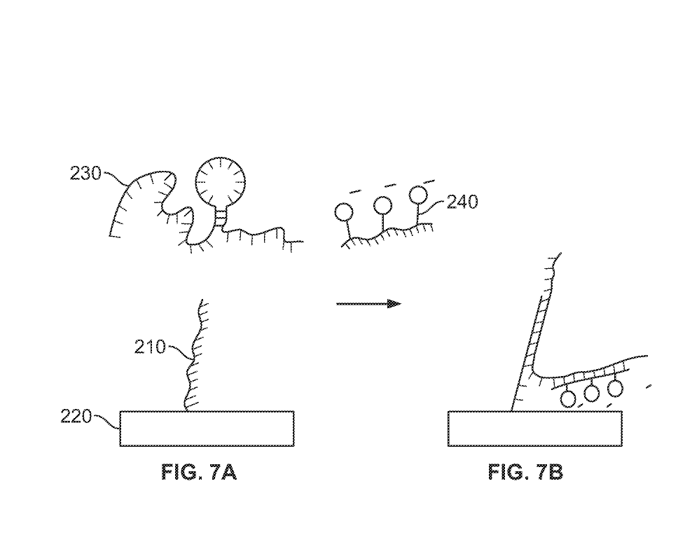

FIGS. 6A and 6B show another embodiment involving the enzymatic extension of a helper oligo. The helper oligo may be designed to only partially hybridize with target 190, leaving a tail 180 that is unhybridized. An enzyme 165, such as phi29 polymerase, and a single-stranded circular template 170 that is at least partially complementary to tail 180 may then be contacted with the complex, as shown in FIG. 6B. FIG. 6C shows the resultant product in which tail 180 is continuously extended around circular template 170, displacing the original hybridization between tail 180 and circular template 170. The result is product 185, which increases the amount of charge localized near the electrode, and thus enhances the detected signal.

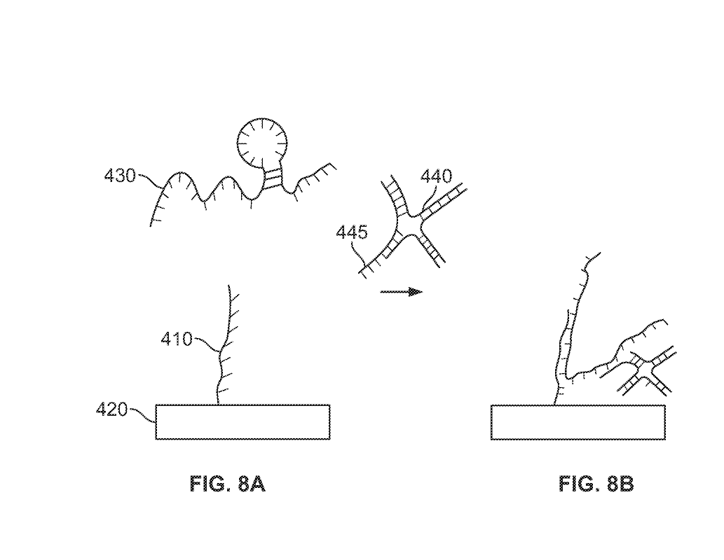

FIGS. 7A and B illustrate the use of a tagged helper oligo 240. Probe 210 is bound to electrode 220. A sample containing target 230 is contacted with helper oligo 240, which is modified to included charged moieties. The moieties may be, for example, covalently attached chemical species, nanoparticles, or any other suitable moiety, combination thereof, and any suitable number of moieties. After a complex is formed by hybridization between probe 210, target 230, and helper oligo 240, a suitable detection method described herein may be used to measure the localized charge near electrode 220.

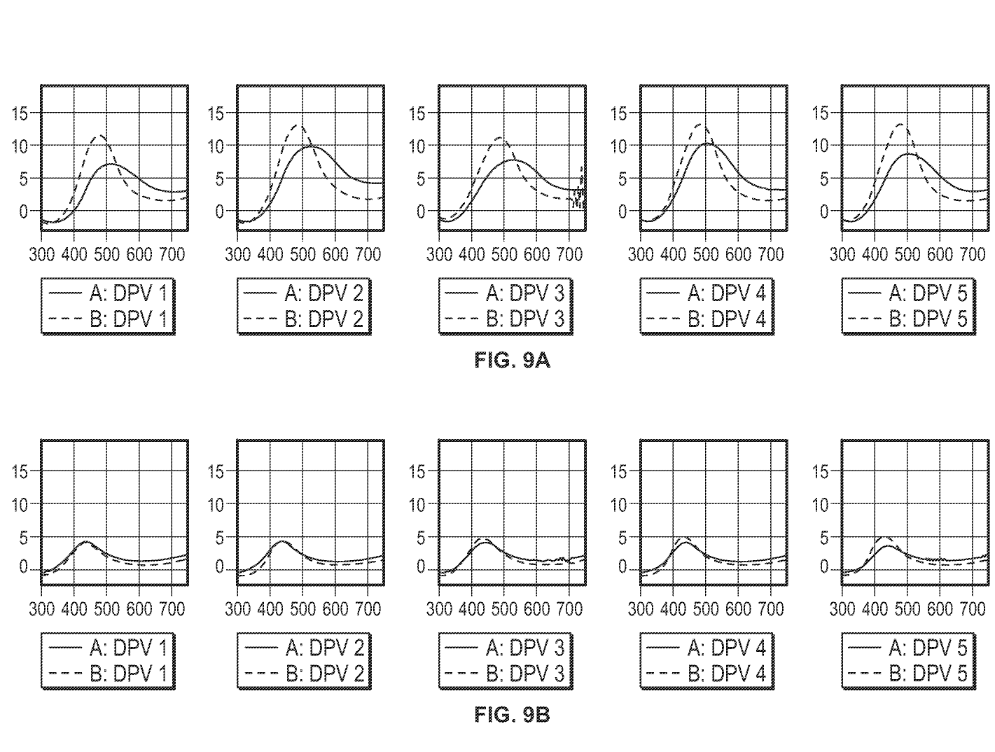

FIGS. 8A and B illustrate the use of a helper oligo 445 partially hybridized with a branched oligonucleotide structure 440 (forming a “branched helper oligo”). Probe 410 is bound to electrode 420. A sample containing target 430 is contacted with helper oligo 445, which is hybridized to structure 440. Structure 440 may be any suitable structure, such as X-shaped, Y-shaped, T-shaped, dendrimer-shaped, linear, or any combination thereof. After a complex is formed by hybridization between probe 410, target 430, and helper oligo 445, a suitable detection method described herein may be used to measure the localized charge near electrode 220 due to the presence of charged structure 440.

To illustrate an example embodiment, a CT OmcA mRNA sequence may be chosen as the target, and have the sequence:

(SEQ ID NO: 1)
ATGAAAAAAAC[[TGCTTTACTCGCTGCTTTATGTAGTGTTGT]]TTC
[TTTAAGTAGTTGTTGTCGTA]TCGTTGACTGTTGCTTCGAAGATCCA
TGCGCACCTATCCAATGTTCACCTTGTGAATCTAAGAAGAAAGACGTA
GACGGTGGTTGCAACTCTTGTAACGGGTATGTCCCAGCTTGCAAACCT
TGCGGAGGGGATACGCACCAAGATGCTAAACATGGCCCTCAAGCTAGA
GGAATTCCAGTTGACGGCAAATGCAGACAATG

A suitable probe sequence may be designed to be complementary to the portion of the CT OmcA mRNA sequence above enclosed in single brackets:

TACGACAACAACTACTTAAA
(sequence #: PP67; SEQ ID NO: 2)

A complementary helper oligo of the following sequence may be designed to bind to the double-bracketed portion of the CT sequence listing above:

ACAACACTACATAAAGCAGCGAGTAAAGCA
(sequence #: 30.3; SEQ ID NO: 3)

The helper oligo may be designed to hybridize to the target such that a terminal end of the helper oligo has at least a 3-base separation from a terminal end of the hybridized probe when both the probe and helper oligo are hybridized to the same target. This separation may be close enough to make the target sequence accessible to the probe, but far enough to prevent steric hindrance between the helper oligo and the probe. In some embodiments, the helper oligo sequence is designed to be 30 bases in length or longer up until 200 bases in length.

In an illustrative protocol to prepare a biosensor, an electrode (including any of the types of electrodes described herein) may be modified with a probe by depositing a 500 nM probe solution (in 10 μM TCEP, 20% CAN, 50 mM NaCl, 0.05% Tween-20) on the electrode and incubating for 2 hours. A solution of mercaptohexanol (MCH) is then added to the deposited solution, bringing the MCH up to a concentration of 250 nMA. After incubating for 16 hours at room temperature, a suitable washing step may be performed, such as washing with 0.1×PBS buffer. A backfill solution of 1 mM mercaptohexanol (MCH) in 0.1×PBS is then contacted with the electrode and incubated for 60 minutes at room temperature.

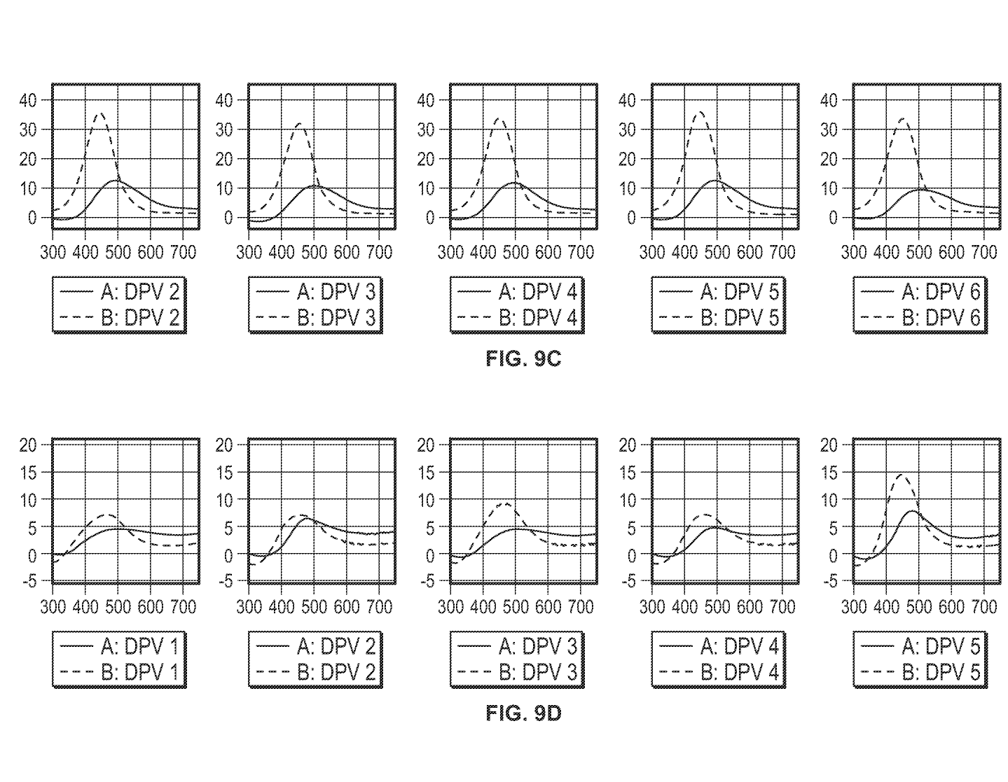

To test the system, 50 nM of the helper oligos (sequence #: 30.3) was mixed with 10⁵ lysed CT. A volume of 50 μL of target solution was placed on the electrode and incubated at 30° C. for 20 minutes. FIGS. 9A-D show the results of electrochemical detection under a variety of conditions. In each case, five individual scans of an electrode were performed using an internal reference and counter electrode prior to the hybridization. FIG. 9A shows an increased signal as a result of CT at 10⁵ (high peak) with no helper oligo, relative to a control (low peak). FIG. 9B corresponds to a blank with 50 nM helper oligo 30.3 only, indicating no substantial change in signal. FIG. 9C corresponds to 10⁵ CT with 50 nM helper oligo 30.3, indicating a signal that is substantially greater than what was measured without the helper oligo in FIG. 9A. FIG. 9D corresponds to a dummy target (10⁵ lactobacillus) with 50 nM helper oligo 30.3, indicating no substantial change except for what appears to be non-specific binding in the fifth replicate.

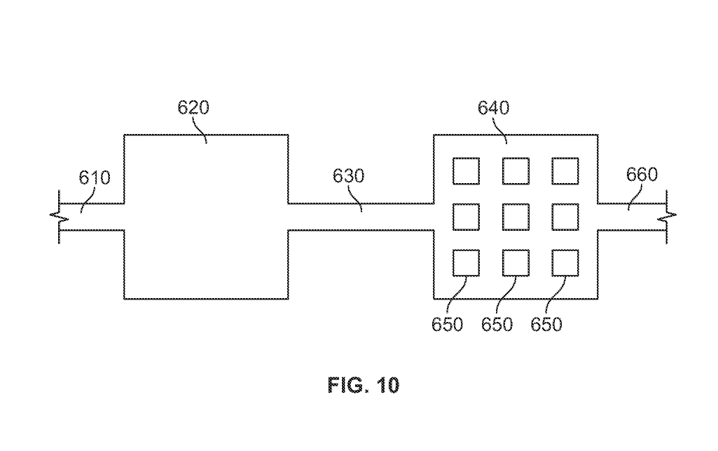

FIG. 10 shows a schematic of sample chambers within a biosensing device. Inlet 610 allows a liquid sample containing or suspected of containing a target to flow into chamber 620. Pressure control between inlet 610 and outlet 660 can be used to direct the direction of sample flow and the duration for which it is in a particular chamber. In chamber 620, the sample will come into contact with helper oligos, catalytic reagents, or other suitable components that facilitate the electrochemical detection of a target. In some embodiments, the helper oligo may exist in a dried state located within chamber 620, but become reconstituted upon contact with the liquid sample. Channel 630 links chamber 620 to chamber 640. Chamber 640 contains electrodes 650 to which probes are bound. In some embodiments, each electrode may have a unique species of probe attached. While the sample is inside chamber 640, electrochemical measurements may be performed using any suitable method described herein. After the measurements are performed, the sample can exit through channel 660.

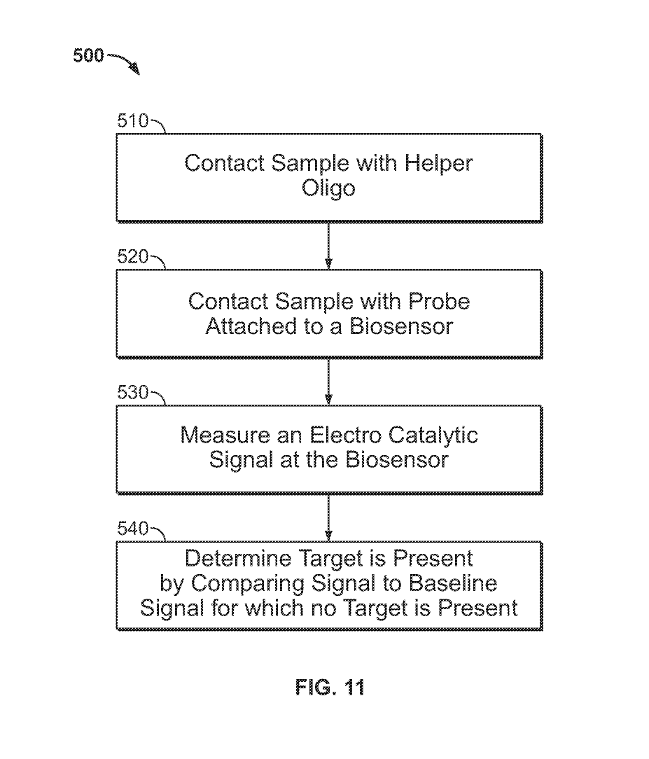

FIG. 11 shows an illustrative process 500 by which a single target could be detected using helper oligos. The process begins at step 510, in which a sample containing or suspected of containing a target is contacted with a helper oligo. This may occur in a separate chamber of a biosensor or separately from the biosensor. The sample may be a liquid or fluid sample including any suitable combination of one or more molecules such as tissue, cells, proteins, fluid, genetic material, bacterial matter or viral matter, plant matter, animal matter, cultured cells, or other organisms or hosts. The sample may contain biological markers indicative of a particular disease or pathogen, as will be described in greater detail below. The sample may be loaded manually by a pipette, automatically flowed into the chamber using microfluidic or macrofluidic channels, or using any other suitable method. In step 520, the sample is contacted with a probe attached to the biosensor. Biosensor preparation may include any suitable pre-processing or preparation steps such as assembling an electrochemical detector from a component kit or functionalizing the electrodes with biosensor probes.

At step 530, an electrochemical signal is measured at the biosensor. The signal may be a current or voltage of any suitable waveform, including DC, AC, square waves, triangle waves, sawtooth waves, decreasing exponentials, or any other signal capable of producing a response signal in response to a biomolecular stimulus, such as nucleic acid hybridization. In some implementations, the response signal is produced in response to an electrochemical reaction that occurs in response to the biomolecular stimulus.

At step 540, a determination is made, based on the response signal, as to whether a target marker is present in the sample. Any suitable detection mechanism may be used, including, for example, determining whether the amplitude of the response signal exceeds a particular threshold, and concluding that the target is present or absent in the sample based on the comparison. In some implementations, a baseline signal is measured under similar measurement conditions for which it is known that no target is present (as a control), and the baseline signal may be subtracted from the signal measured when the target is believed to be present. After the signal is corrected for the baseline, it is compared to a particular threshold to determine if the target marker is present. The determination may be made using any suitable processing circuitry coupled to the multiplexed detection unit. In some implementations, a separate measurement of the sample may be performed without the helper oligo being present. This measurement may be compared to or subtracted from the sample in which the helper oligo was present in order to correct for non-specific binding of the target to the probe.

In some implementations, the electrochemical detector is fabricated as a standalone chip with a plurality of pins. The pins may be arranged in any suitable fashion to interface with an external processor for which quantitative determinations, such as threshold comparisons, can be performed. The electrochemical detector includes a readout device that generates an indicator to communicate the results of the detection. The readout device may be any suitable display device, such as LED indicators, a touch-activated display, an audio output, or any combination of these. Any suitable mechanism for indicating the presence or absence of the target may be used. For example, the indicator may include an amplitude of the first response signal, a concentration of the first target marker determined based on the first response signal, a color-coded indicator selected based on the response signal, a symbol selected based on the a particular response signal, a graphical representation of the response signal over a plurality of values for a corresponding input signal, and any suitable combination thereof.

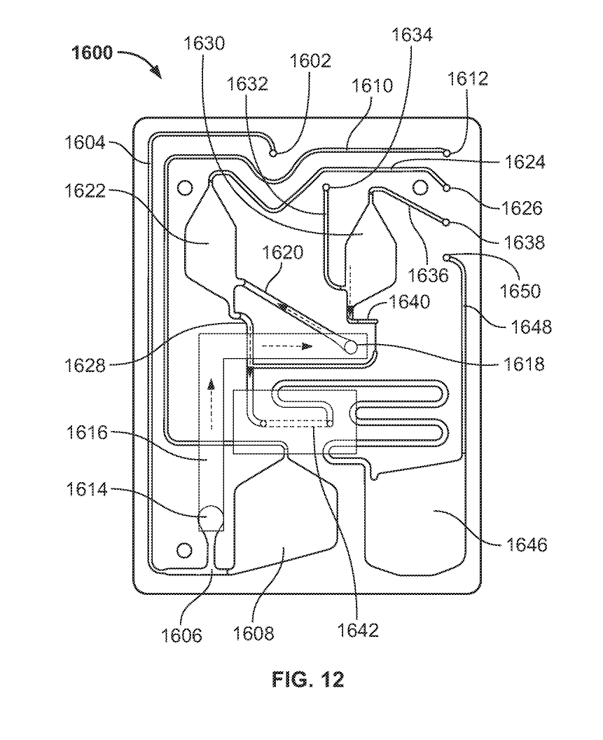

The systems, devices, methods, and all embodiments described above may be incorporated into a cartridge to prepare a sample for analysis and perform a detection analysis. FIG. 12 depicts a cartridge system 1600 for receiving, preparing, and analyzing a biological sample. For example, cartridge system 1600 may be configured to remove a portion of a biological sample from a sample collector or swab, transport the sample to a lysis zone where a lysis and fragmentation procedure are performed, and transport the sample to an analysis chamber for determining the presence of various markers and to determine a disease state of a biological host.

The system 1600 includes ports, channels, and chambers. System 1600 may transport a sample through the channels and chambers by applying fluid pressure, for example, with a pump or pressurized gas or liquids. In certain embodiments, ports 1602, 1612, 1626, 1634, 1638, and 1650 may be opened and closed to direct fluid flow. In use, a sample is collected from a patient and applied to the chamber through port 1602. In certain approaches, the sample is collected into a collection chamber or test tube, which connects to port 1602. In practice, the sample is a fluid, or fluid is added to the sample to form a sample solution. In certain approaches, additional reagents are added to the sample. The sample solution is directed through channel 1604, past sample inlet 1606, and into degassing chamber 1608 by applying fluid pressure to the sample through port 1602 while opening port 1612 and closing ports 1626, 1634, 1638, and 1650. The sample solution enters and collects in degassing chamber 1608. Gas or bubbles from the sample solution also collect in the chamber and are expelled through channel 1610 and port 1612. If bubbles are not removed, they may interfere with processing and analyzing the sample, for example, by blocking flow of the sample solution or preventing the solution from reaching parts of the system, such as a lysis electrode or sensor. In certain embodiments, channel 1610 and port 1612 are elevated higher than degassing chamber 1608 so that the gas rises into channel 1610 as chamber 1608 is filled. In certain approaches, a portion of the sample solution is pumped through channel 1610 and port 1612 to ensure that all gas has been removed.

After degassing, the sample solution is directed into lysis chamber 1616 by closing ports 1602, 1634, 1638, and 1650, opening port 1626, and applying fluid pressure through port 1612. The sample solution flows through inlet 1606 and into lysis chamber 1616. In certain approaches, system 1600 includes a filter 1614. Filter 1614 may be a physical filter, such as a membrane, mesh, or other material to remove materials from the sample solution, such as large pieces of tissue, which could clog the flow of the sample solution through system 1600. Lysis chamber 1616 may be similar to lysis chamber 1200 or lysis chamber 1310 described previously. When the sample is in lysis chamber 1616, a lysis procedure, such as an electrical lysis procedure as described above, may be applied to release analytes into the sample solution. For example, the lysis procedure may lyse cells to release nucleic acids, proteins, or other molecules which may be used as markers for a pathogen, disease, or host. In certain approaches, the sample solution flows continuously through lysis chamber 1616. Additionally or alternatively, the sample solution may be agitated while in lysis chamber 1616 before, during, or after the lysis procedure. Additionally or alternatively, the sample solution may rest in lysis chamber 1616 before, during, or after the lysis procedure.

Electrical lysis procedures may produce gases (e.g., oxygen, hydrogen), which form bubbles. Bubbles formed from lysis may interfere with other parts of the system. For example, they may block flow of the sample solution or interfere with hybridization and sensing of the marker at the probe and sensor. Accordingly, the sample solution is directed to a degassing chamber or bubble trap 1622. The sample solution is directed from lysis chamber 1616 through opening 1618, through channel 1620, and into bubble trap 1622 by applying fluid pressure to the sample solution through port 1612, while keeping port 1626 open and ports 1602, 1634, 1638, and 1650 closed. Similar to degassing chamber 1608, the sample solution flows into bubble trap 1622 and the gas or bubbles collect and are expelled through channel 1624 and port 1626. For example, channel 1624 and port 1626 may be higher than bubble trap 1622 so that the gas rises into channel 1624 as bubble trap 1622 is filled. In certain approaches, a portion of the sample solution is pumped through channel 1624 and port 1626 to ensure that all gas has been removed.

After removing the bubbles, the sample solution is pumped through channel 1628 and into analysis chamber 1642 by applying fluid pressure through port 1626 while opening port 1650 and closing ports 1602, 1612, 1634, and 1638. Analysis chamber 1642 is similar to previously described analysis chambers, such as chambers 400, 500, 600, 700, 800, 900, 1000, 1100, and 1306. Analysis chamber 1642 includes sensors, such as a pathogen sensor, host sensor, and non-sense sensor as previously described. In certain approaches, the sample solution flows continuously through analysis chamber 1642. Additionally or alternatively, the sample solution may be agitated while in analysis chamber 1642 to improve hybridization of the markers with the probes on the sensors. In certain approaches, system 1600 includes a fluid delay line 1644, which provides a holding space for portions of the sample during hybridization and agitation. In certain approaches, the sample solution sits idle while in analysis chamber 1642 as a delay to allow hybridization.

System 1600 includes a reagent chamber 1630, which holds electrochemical reagents, such as transition metal complexes Ru(NH3)₆³⁺ and Fe(CN)₆³⁻, for electrochemical detection of markers in the sample solution. In certain approaches, the electrochemical reagents are stored in dry form with a separate rehydration buffer. For example, the rehydration buffer may be stored in a foil pouch above rehydration chamber 1630. The pouch may be broken or otherwise opened to rehydrate the reagents. In certain approaches, a rehydration buffer may be pumped into rehydration chamber 1630. Adding the buffer may introduce bubbles into chamber 1630. Gas or bubbles may be removed from rehydration chamber 1630 by applying fluid pressure through port 1638, while opening port 1634 and closing ports 1602, 1624, 1626, and 1650 so that gas is expelled through channel 1630 and port 1634. Similarly, fluid pressure may be applied through port 1634 while opening port 1638. After the sample solution has had sufficient time to allow the markers to hybridize to sensor probes in the analysis chamber, the hydrated and degassed reagent solution is pumped through channel 1640 and into analysis chamber 1642 by applying fluid pressure through port 1638, while opening port 1650 and closing all other ports. The reagent solution pushes the sample solution out of analysis chamber 1642, through delay line 1644, and into waste chamber 1646 leaving behind only those molecules or markers which have hybridized at the probes of the sensors in analysis chamber 1642. In certain approaches, the sample solution may be removed from the cartridge system 1600 through channel 1648, or otherwise further processed. The reagent solution fills analysis chamber 1642. In certain approaches, the reagent solution is mixed with the sample solution before the sample solution is moved into analysis chamber 1642, or during the flow of the sample solution into analysis chamber 1642. After the reagent solution has been added, an electrochemical analysis procedure to detect the presence or absence of markers is performed as previously described.

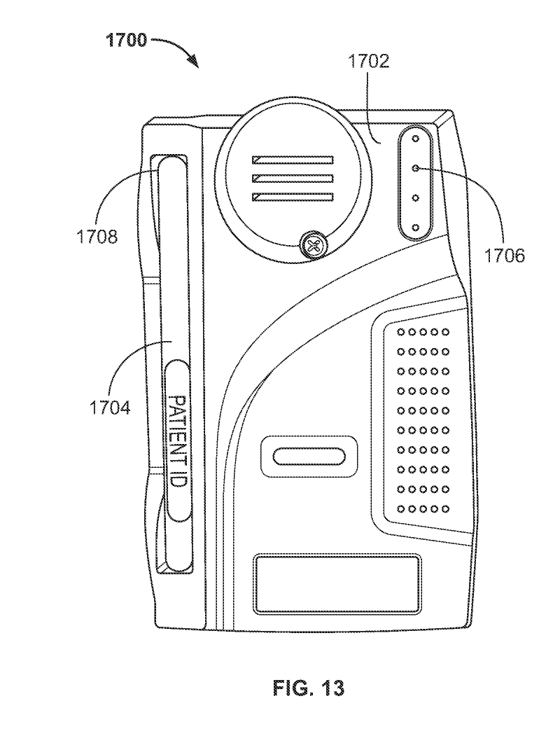

FIG. 13 depicts an embodiment of a cartridge for an analytical detection system. Cartridge 1700 includes an outer housing 1702, for retaining a processing and analysis system, such as system 1600. Cartridge 1700 allows the internal processing and analysis system to integrate with other instrumentation. Cartridge 1700 includes a receptacle 1708 for receiving a sample container 1704. A sample is received from a patient, for example, with a swab. The swab is then placed into container 1704. Container 1704 is then positioned within receptacle 1708. Receptacle 1708 retains the container and allows the sample to be processed in the analysis system. In certain approaches, receptacle 1708 couples container 1704 to port 1602 so that the sample can be directed from container 1704 and processed though system 1600. Cartridge 1700 may also include additional features, such as ports 1706, for ease of processing the sample. In certain approaches, ports 1706 correspond to ports of system 1600, such as ports 1602, 1612, 1626, 1634, 1638, and 1650 to open or close to ports or apply pressure for moving the sample through system 1600.

Cartridges may use any appropriate formats, materials, and size scales for sample preparation and sample analysis. In certain approaches, cartridges use microfluidic channels and chambers. In certain approaches, the cartridges use macrofluidic channels and chambers. Cartridges may be single layer devices or multilayer devices. Methods of fabrication include, but are not limited to, photolithography, machining, micromachining, molding, and embossing.

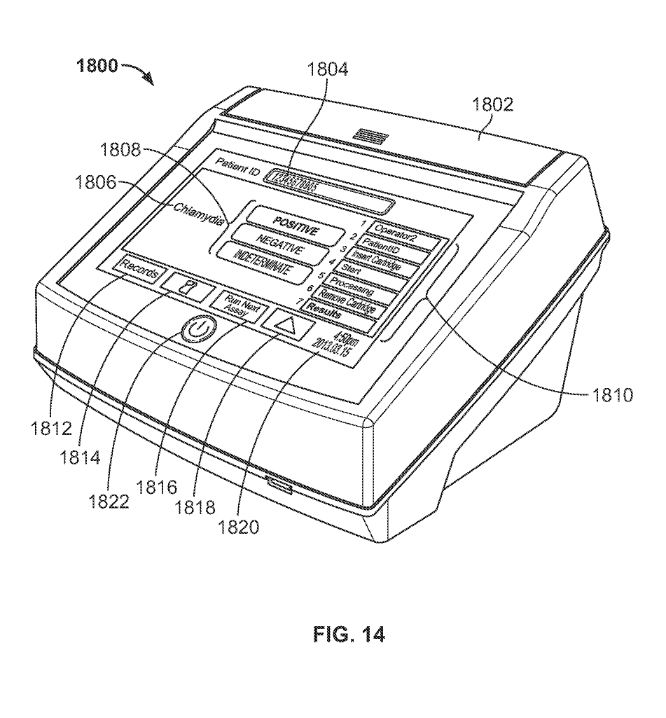

FIG. 14 depicts an automated testing system to provide ease of processing and analyzing a sample. System 1800 may include a cartridge receiver 1802 for receiving a cartridge, such as cartridge 1700. System 1800 may include other buttons, controls, and indicators. For example, indicator 1804 is a patient ID indicator, which may be typed in manually by a user, or read automatically from cartridge 1700 or cartridge container 1704. System 1800 may include a “Records” button 1812 to allow a user to access or record relevant patient record information, “Print” button 1814 to print results, “Run Next Assay” button 1818 to start processing an assay, “Selector” button 1818 to select process steps or otherwise control system 1800, and “Power” button 1822 to turn the system on or off Other buttons and controls may also be provided to assist in using system 1800. System 1800 may include process indicators 1810 to provide instructions or to indicate progress of the sample analysis. System 1800 includes a test type indicator 1806 and results indicator 1808. For example, system 1800 is currently testing for Chlamydia as shown by indicator 1806, and the test has resulted in a positive result, as shown by indicator 1808. System 1800 may include other indicators as appropriate, such as time and date indicator 1820 to improve system functionality.

The foregoing is merely illustrative of the principles of the disclosure, and the systems, devices, and methods can be practiced by other than the described embodiments, which are presented for the purposes of illustration and not of limitation. It is to be understood that the systems, devices, and methods disclosed herein, while shown for use in detection systems for bacteria, and specifically, for Chlamydia Trachomatis, may be applied to systems, devices, and methods to be used in other applications including, but not limited to, detection of other bacteria, viruses, fungi, prions, plant matter, animal matter, protein, RNA sequences, DNA sequences, as well as cancer screening and genetic testing, including screening for genetic disorders.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented. All references cited are hereby incorporated by reference herein in their entireties and made part of this application.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. A method for detecting a target, the method comprising:
contacting a sample with a helper oligonucleotide capable of forming a first complex with a target in the sample;
contacting the sample with a probe affixed to a biosensor, wherein the probe is capable of forming a second complex with the first complex; and
measuring a first electrochemical signal at the biosensor, wherein the first electrochemical signal is indicative of the presence of the second complex.

2. The method of claim 1, further comprising determining that the target is present in the sample by comparing the first electrochemical signal to a second electrochemical signal measured absent the presence of the second complex.

3. The method of claim 1, wherein the first electrochemical signal is generated by charge accumulation at the surface of the biosensor in response to the formation of the second complex.

4. The method of claim 1, further comprising contacting the sample with an enzyme, wherein the helper oligonucleotide is capable of being enzymatically extended when the second complex is formed.

5. The method of claim 4, further comprising contacting the sample with a circular template, wherein a portion of the helper oligonucleotide is capable of binding to the circular template.

6. The method of claim 5, wherein the helper oligonucleotide is capable of being enzymatically extended by rolling circle amplification when the portion of the helper oligonucleotide is bound to the circular template.

7. The method of claim 1, wherein the helper oligonucleotide is tagged with a charged moiety.

8. The method of claim 1, wherein the helper oligonucleotide is partially hybridized to a branched oligonucleotide structure.

9. The method of claim 1, wherein the helper oligonucleotide is between 30 and 200 bases in length.

10. The method of claim 1, wherein a terminal end of the helper oligonucleotide is at least 3 bases away from a terminal end of the probe when the second complex is formed.

11. The method of claim 1, wherein the formation of the second complex is more thermodynamically favorable than a binding of the target to the probe in the absence of the helper oligonucleotide.

12. The method of claim 1, wherein, when the second complex is formed, a terminal end of the helper oligonucleotide is closer to a surface-bound terminal end of the probe than to a non-surface-bound terminal end of the probe.

13. The method of claim 1, wherein the helper oligonucleotide is between 30 and 100 bases in length.

14. A point-of-care diagnostic device configured to perform the method of claim 1.

15. A biosensor comprising:
a solid support;
a probe affixed to the solid support;
a first chamber for contacting a sample with a helper oligonucleotide;
a second chamber for contacting the sample with the probe;
wherein:
the first chamber is operatively connected to the second chamber by a flow channel;
the probe is capable of forming a complex comprising the probe, the helper molecule, and a target suspected of being in the sample; and
control circuitry operably coupled to the solid support, wherein the control circuitry is configured to detect the presence of the complex.

16. A point-of-care diagnostic device including the biosensor of claim 15.


SEQUENCE LISTING (3 sequences)

SEQ ID NO: 1 - DNA, 266 bp, Artificial Sequence
  Description of Artificial Sequence Synthetic polynucleotide
atgaaaaaaactgctttactcgctgctttatgtagtgttgtttctttaagtagttgttgtcgtatcgttgactgttgcttcgaagatccatgcgcacctatccaatgttcaccttgtgaatctaagaagaaagacgtagacggtggttgcaactcttgtaacgggtatgtcccagcttgcaaaccttgcggaggggatacgcaccaagatgctaaacatggccctcaagctagaggaattccagttgacggcaaatgcagacaatg

SEQ ID NO: 2 - DNA, 20 bp, Artificial Sequence
  Description of Artificial Sequence Synthetic oligonucleotide
tacgacaacaactacttaaa

SEQ ID NO: 3 - DNA, 30 bp, Artificial Sequence
  Description of Artificial Sequence Synthetic oligonucleotide
acaacactacataaagcagcgagtaaagca